(19) United States
(12) Patent Application Publication
(10) Pub. No.: US 2017/0245830 A1
(43) Pub. Date: Aug. 31, 2017

(54) SYSTEM AND PROCESS FOR ULTRASONIC DETERMINATION OF LONG BONE ORIENTATION

(71) Applicant: THINK SURGICAL, INC., Fremont, CA (US)

(72) Inventors: Nathan A. NETRAVALI, Palo Alto, CA (US); Micah FORSTEIN, Fremont, CA (US); Joel ZUHARS, Fremont, MI (US); In K. MUN, Fremont, CA (US)

(73) Assignee: Think Surgical, Inc., Fremont, CA (US)

(21) Appl. No.: 15/511,101

(22) PCT Filed: Sep. 21, 2015
(86) PCT No.: PCT/US2015/051165

§ 371 Date: Mar. 14, 2017

Related U.S. Application Data
(60) Provisional application No. 62/052,871, filed on Sep. 19, 2014.

(51) Int. Cl. A61B 8/08; A61B 34/10; A61B 8/00

(52) U.S. Cl. CPC A61B 8/0875; A61B 8/4477; A61B 8/4245; A61B 8/5207; A61B 34/10

(57) ABSTRACT

Systems and process are provided for a tool to create a registration matrix for a bone in three dimensional workspaces relative to a system using the absence of an ultrasonic reflection from an ultrasonic sensor attached to a tracking system to build the registration matrix model. The system has hardware and software that creates an absence of ultrasonic reflection, and utilizes the information to identify the location and orientation of a set of conic volumes in order to build an estimate of the position and orientation of the bone relative to the base system, and to use the position and orientation changes to update the registration and to detect bone motion. Embodiments of invention provide a process of registering part or all of a bone without directly touching the bone using ultrasound, and as a result support a non-invasive or less-invasive approach to bone registration and bone motion detection for a system.

### CROSS-REFERENCE TO RELATED APPLICATION

This application claims priority of U.S. Provisional Patent Application Ser. No. 62/052,871 filed Sep. 19, 2014, which is incorporated herein by reference.

### FIELD OF THE INVENTION

The present invention in general relates to systems and process of three dimensional ultrasonic imaging of hard tissue and, more particularly, to a system and process for ultrasonic determination of bone orientation.

### BACKGROUND OF THE INVENTION

Ultrasound is an oscillating sound pressure wave with a frequency greater than the upper limit of the human hearing range. Ultrasound devices operate with frequencies from 20 kHz up to several gigahertz. Ultrasonic devices are used to detect objects and measure distances.

Ultrasonic imaging (sonography) is used in both veterinary medicine and human medicine. Ultrasonic bone imaging is advantageous to traditional radio-graphic images (e.g., X-Ray or CT scan) of bone tissue as a these methods require exposing a patient to radiation. Significant amounts of radiation are required in order to produce three dimensional (3D) images using these techniques. Further, the equipment required for the practice of these techniques is typically expensive and of limited portability. Ultrasound offers a promising alternative to these prior art methods of bone imaging.

Currently, robotic assisted surgical methodology requires the surgeon to digitize several points on the ends of the bone being operated on, and then three points in the shaft on the long bone. Because there are no incisions made this far down the shaft, the surgeon must percutaneously locate these points in the shaft which presents added risk since there is no visibility and there is a risk of hitting a nerve or artery. Thus, there exists a need to eliminate the need for these percutaneous incision points in robotic surgery on bones, and for a new non-invasive method for serving the same purpose.

### SUMMARY OF THE INVENTION

A system for ultrasonic determination of bone orientation is provided that includes a base system, a tracking system, and a plurality of ultrasonic sensors. The system creates a registration matrix model for a bone in a three dimensional workspace relative to the base system using an absence of an ultrasonic reflection from the one or more ultrasonic sensors attached to the tracking system to build the registration model of the bone. The system utilizes the absence of the ultrasonic reflection to identify a location and orientation of a set of conic volumes in order to build an estimate of the location and orientation of the bone relative to the base system.

A process for ultrasonic determination of bone orientation includes: registering the base system to the bone; attaching the tracking system to the plurality of ultrasonic sensors;

utilizing an algorithm to convert a set of positions and orientations of the ultrasonic sensors to a plurality of markers fixed to the plurality ultrasonic sensors, where a set of three dimensional (3-D) locations of the plurality of markers are attached to the ultrasonic sensors that fix the relative position and orientation of the plurality of markers to the set of positions and orientations of the ultrasonic sensors; and wherein the algorithm uses the set of 3-D locations and the relative orientation and position of the plurality of markers tracked by the tracking system to calculate a second set of positions and orientations of ultrasonic oriented rays or ultrasonic cones generated by the plurality of ultrasonic sensors, where the algorithm uses the second set of positions and orientations to track a third set of positions and orientations of reflected or non-reflective bone within a patient in 3-D space.

A process of preparing for a surgical procedure on a bone within a patient is provided by which the position and orientation of the bone within a measurement volume is calculated relative to a base system. The process includes measuring a distance to the bone in a conic region emanating from an ultrasonic sensor at a distance where the orientation and volume of the conic region is known. The conic region of the ultrasonic sensor is then moved within the measurement volume. A first sweep motion is generated to measure the distance within the conic region that intersects with the solid volume of the bone within the measurement volume. A second sweep motion is generated of the ultrasonic sensor while not measuring the distance of the solid volume of the bone when the solid volume is not in the conic region. The position and orientation of the ultrasonic sensor is determined within the measurement volume during measurement; as are the position and orientation of the measurement volume and the position and orientation of the bone with respect to the base system. The position and orientation of the bone are communicated to a surgeon preparing for the surgical procedure on the bone within the patient. In some inventive embodiments, the measurement volume is determined by totaling the conic region for all collected data and subtracting from the conic region for all collected data, the conic region of measure from the measurement volume when no distance to a solid volume is measured to determine a remaining volume. The remaining volume is utilized to locate and orient a surface model that represents the solid volume of the bone that was located within the measurement volume during measurement. The position and orientation of the surface model of the solid volume within the measurement volume is then calculated relative to the base system.

In some specific inventive embodiments, the collected measurement data is determined by moving a sweeping beam of the ultrasonic sensor along a shaft of the bone from different orientations about the shaft; and calculating a complete result by sweeping around the bone by 180+2* cone angle during the data collection process or a partial result is calculated by using only two orientations about the shaft of the bone that are ninety degrees apart.

### BRIEF DESCRIPTION OF THE DRAWINGS

The present invention is further detailed with respect to the following drawings that are intended to show certain aspects of the present invention, but should not be construed as a limit on the practice of the present invention.

FIG. 1 illustrates a perspective view of an ultrasonic system for the determination of bone orientation in a patient according to an embodiment of the invention; and

FIG. 2 is a block diagram of an ultrasonic system for the determination of bone orientation in a patient according to an embodiment of the invention.

### DESCRIPTION OF THE INVENTION

The present invention has utility to create a registration matrix for a bone in three dimensional workspaces relative to a system using the absence of an ultrasonic reflection from an ultrasonic sensor attached to a tracking system as a process to build the registration model. The system has hardware and software that creates an absence of ultrasonic reflection, and utilizes the information to identify the location and orientation of a set of conic volumes in order to build an estimate of the position and orientation of the bone relative to the base system, and to use the position and orientation changes to update the registration and to detect bone motion. Embodiments of invention provide a process of registering part or all of a bone without directly touching the bone, and as a result support a non-invasive or less-invasive approach to bone registration and bone motion detection for a system.

Advantages of embodiments of the inventive system and process for ultrasonic determination of long bone orientation include the ability to register the location and orientation of the bone without the need for additional incisions or percutaneous probe punctures. Current robotic assisted surgical methodology requires the surgeon to digitize several points on the ends of the bone being operated and then three points in the shaft on the long bone. Because there are no incisions made this far down the shaft, the surgeon must percutaneously locate these points in the shaft which presents added risk since there is no visibility and there is a risk of hitting a nerve or artery. Embodiments of the invention eliminate the need for these percutaneous points and present a new non-invasive process for serving the same purpose.

The inventive system utilizes ultrasound and tracking information to indirectly measure a bone location by defining where the bone is not. The novel process results in a definition of the surface model of the bone and a set of conic volumes as a way to register part or all of a bone or detect bone motion relative to a fixed coordinate frame such as a surgical robot or navigation before, after, or during a procedure.

The inventive system includes a base system, a tracking system, and two or more ultrasonic sensors. The base system is registered to a bone to perform functions including, but not limited to, a robotic cutting system and/or a navigation system. The tracking system may be used to determine a point or a series of points in space, which in turn can be fixed upon an object. The tracking system may use technologies such as a mechanical linkage, optical cameras, electromagnetic (EM) sensing, or other forms of tracking technology relying on a combination of markers and cameras. The two or more ultrasonic sensors are capable of capturing a conic ultrasonic reflection from a synchronized ultrasonic source. The ultrasonic sensors are also capable of detection of ultrasonic sound waves, detection of a unidirectional ultrasonic cone while pointing in a specific orientation, detecting a reflection of ultrasonic sound waves off a bone within a human being. Furthermore, the two or more ultrasonic sensors will not detect a bone reflection in the absence of bone. The sensors are therefore capable of detecting and outputting the reflection of bone and the absence of bone, whereupon the transition of a bone reflection to no reflection is indicative of the bone edge.

Included in the system is an attachment mechanism between the ultrasonic sensors and a plurality of tracking system markers such that the sensors can be tracked by the tracking system, with features that include: a process to attach a plurality of ultrasonic sensors and or ultrasonic sources, a process to attach a plurality of tracking system markers in a specific configuration if required, a rigid frame capable of holding the plurality of sensors or markers in a fixed position relative to each other, and a process of relating the relative position of the plurality of sensors or sources to the plurality of markers. Tracking system markers illustratively include:

active markers, such as light emitting diodes; passive reflectors, such as a plastic sphere with a retro-reflective film; a distinct pattern or sequence of shapes, lines or other characters; acousting emitters or reflectors; magnetic emitters or reflectors; accelerometers; gyroscopes; and the like or any combination thereof. Markers may also illustratively include sound channeling shapes such as spheres and tetrahedral; the shape formed from a variety of materials such as stainless steel.

The ultrasonic sensors may also be tracked by attaching the ultrasonic sensor to the distal end of a mechanical tracking device. The mechanical tracking device may be attached to the base system and include a series of linkages. The linkages are connected by joints with encoders that measure the position and orientation of the attached ultrasonic sensors. Calibration techniques are used to relate any position and orientation scanning information received by the ultrasonic sensor to the base system.

In the inventive system, an active or passive process to relocate a plurality of sensors within the operative theater that houses the inventive system may be used including at least one of the following: an active process via a motor, actuator or plurality of motors or actuators that can reposition and or reorient the plurality of sensors, sources and markers in a pre-prescribed way if required; a passive process via a hand held wand that can reposition and or reorient the plurality of sensors, sources and markers in a pre-prescribed way if required; and a combination of active and passive reorientation process as described above.

The system utilizes an algorithm to convert the position and orientation of the ultrasonic sensors to the plurality of markers fixed to the ultrasonic sensors. The 3-D locations of the plurality of markers are attached to the ultrasonic sensors that fix the relative position and orientation of the markers to the position and orientation of the ultrasonic sensors. The algorithm can use the 3-D location and the relative orientation and position of the plurality of markers tracked by the tracking system to calculate the position and orientation of the ultrasonic oriented rays or ultrasonic cones of the sensors. Thereby tracking the position and orientation of the reflected or non-reflective bone within the patient in 3-D space.

In another embodiment, the algorithm utilizes the tracked orientation and position of the ultrasonic sensors to create a solid volume model of the bone by adding the tracked conic regions from all the collected measurement data. Once the conic regions are added, an algorithm within the software calculates a subtraction volume by subtracting the oriented conic volumes with no ultrasonic reflection. The algorithm determines the volume remaining once all of the conic regions with no ultrasonic reflection have been subtracted. A solid volume of the bone remains with the relative position and orientation of the bone in 3-D space as determined from the tracking system and the plurality of tracking markers attached to the ultrasonic sensors. The algorithm then converts the 3D orientation of the surface model into a registration model. The system subsequently utilizes the algorithm to convert the registration model into a registration of the bone surface model relative to the base system.

The portion of the algorithm that converts the registration model into a registration of the bone relative to the base system calculates the position of the bone based upon the transformation of the bone relative to the tracking system and the transformation of the tracking system relative to the base system. The algorithm utilizes the following to calculate the position and orientation of the bone within the measurement volume relative to the base system: a solid volume within a field of measure (a bone); measuring a distance in a conic region emanating from a fixed or mobile source at a distance where the orientation and volume of the conic region is known; moving the conic region of the measurement sensor within the measurement volume; a motion (or sweep) that will cause the sensor to measure the distance within the conic region that intersects with the solid volume within the measurement volume; a motion (or sweep) that will cause the sensor not to measure the distance of the solid volume when solid volume is not in the conic region; determining the position and orientation of the measurement sensor within the measurement volume during measurement; and determining the position and orientation of the measurement volume with respect to the base system. As a result, a surgeon preparing for a surgical procedure on a bone within a patient is informed of the position and orientation of the bone within a measurement volume relative to a base system.

The algorithm based on the above determinations is able to: create a measured volume by adding the conic region for all collected measurement data; subtract the conic region of measure from the measured volume when no distance to a solid volume is measured; determine the volume remaining within the measured volume once the all of the conic regions where no distance was measured are subtracted; utilize the remaining calculated volume to locate and orient a surface model that represents the solid volume that was located within the measurement volume during measurement; and calculate the position and orientation of the surface model of the solid volume within the measurement volume relative to the base system.

The inventive algorithm provides the ability to: locate and orient a surface model within the measurement volume; find a partial position and orientation of the surface model within the measurement volume; find the surface model of a bone shaft by moving the sweeping beam along the shaft from different orientations about the shaft; and calculate a complete result by sweeping around the object by 180+2* cone angle during the data collection process.

The algorithm can calculate a partial result by using only two orientations about the shaft that are ninety degrees apart, effectively calculating the last 2 degrees of freedom that define the bone shaft position, considering that the midpoint of the measured result should be the same as the midpoint of the surface model.

Embodiments of the inventive system have the ability to collect and compute the registration automatically. The system may update the bone to base system while the tracking system and ultrasonic systems are active. The system may also have the ability to calculate any change in the computed registration to determine bone motion. The system will use any change in registration as an indication of bone motion. Specific embodiments of the system have the ability to determine the position and orientation of clinically relevant anatomic landmarks relative to the base system, as well as to determine and differentiate between the base system orientation and the registered bone.

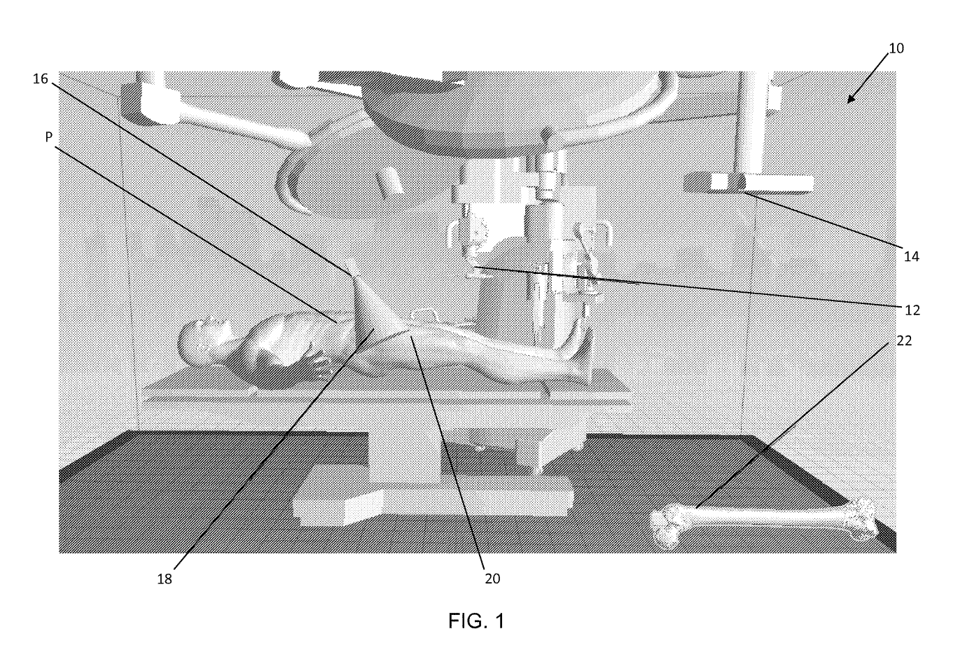

Referring now to figures, FIG. 1 illustrates a perspective view of an ultrasonic system shown generally at 10 for the determination of bone orientation in a patient P according to an embodiment of the invention. A base system 12 has a tracking system 14 that tracks ultrasonic sensors 16 that generate conic volumes 18 that sweep the patient P to generate a three dimensional bone surface model (shown as 22) of the actual bone 20 of the patient P. In a particular embodiment, the ultrasonic sensors generate conic volume 18 that sweep the patient P to identify a position and orientation of a long bone of the patient P using the methods described herein.

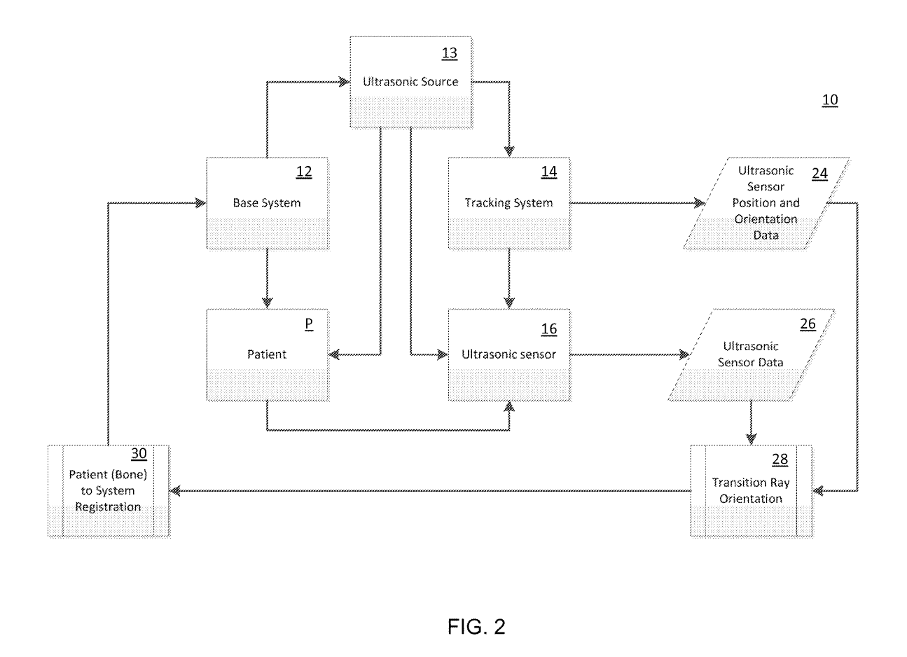

FIG. 2 is a schematic block diagram of ultrasonic system 10 for the determination of bone orientation in a patient P according to an embodiment of the invention. A base system 12 has an ultrasonic source 13 that is supplied to ultrasonic sensors 16 and to the tracking system 14. Based on information obtained via the tracking system 14, ultrasonic sensor position and orientation data 24 may be obtained. The ultrasonic sensor data 26 from the ultrasonic sensor 16 scan of the patient P is combined with the ultrasonic sensor position and orientation data 24 to obtain transition ray orientation 28 that is used to determine the patient bone to system registration 30.

In a particular embodiment, base tracking markers are attached to the base system to track the position and orientation of the base system by the tracking system. An ultrasonic source, independent of the base system, is attached to the ultrasonic sensors. Ultrasound tracking markers are attached to the ultrasonic source/ultrasonic sensor combination and calibrated so the position and orientation of the scanning information is known with respect to the ultrasound tracking markers. The tracking system can then accurately track the position and orientation of scanning information from the ultrasound sensors via the ultrasound tracking markers. The patient bone to base system registration is calculated from the tracked scanning information and the tracked base system.

### EXAMPLES

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Example 1

In total hip arthroplasty, a computer assisted surgical device is used to aid a surgeon in planning and executing the preparation of the femoral canal to receive a femoral implant. The surgical device has an end mill that precisely drills the femoral canal based on the surgeon's pre-operative plan. Once the plan is loaded into the surgical device and the patient's femoral head and neck are exposed as in traditional total hip arthroplasty, the coordinate frames of the surgical device and the anatomy need to be aligned or registered so the end mill knows the position and orientation of the bone to ensure the cavity is accurately milled. A 3-D surface model of a patient's bone is generated from pre-operative imaging data to help plan the case but also to help facilitate and increase the accuracy of registration.

An optical tracking system is used to track the position and orientation of multiple objects in the operating room by attaching optical tracking arrays consisting of passive or active markers that are tracked by two or more cameras. A probe with a tracking array is used to collect a set of points on the exposed femoral head and neck to obtain some initial registration points to align the coordinate frames of the surgical device to the bone. However, to increase the accuracy of the registration, points near the mid-shaft of the bone need to be collected to properly align the patient's bone with the 3-D surface model. Traditionally, the surgeon could percutaneously collect the points through the skin on the bone. Instead, ultrasonic sensors are used to define the long bone axis to complete the registration. The ultrasonic sensors have a tracking array fixed to the sensors where two or more cameras can calculate the position and orientation of the sensors in 3-D space as well as the position and orientation of the ultrasonic beam or conic volume reflected or not reflected from the bone.

The ultrasonic sensor is swept along the mid-shaft of the bone whereupon the bone edges can be determined and located in 3-D space by way of the tracking system. The probe is then moved 90° about the shaft and similarly swept along the long axis of the bone. The ultrasonic probe and sensor are capable of detecting the reflectance of bone and the non-reflectance of bone to obtain a plurality of points, bone volume, or bone outline that can indicate the position and orientation of the bone within the patient without collecting the points percutaneously. Only the two orientations are needed as they are the last two degrees of freedom that define the bone shaft position, considering that the midpoint of the measured result should be the same as the midpoint of the surface model. The collected points, bone volume, or bone outline can be used to automatically register the patient's bone to the 3-D surface model and correspondingly to the surgical device using an algorithm that calculates the position of the bone based upon the transformation of the bone relative to the tracking system and the transformation of the tracking system relative to the base system.

### Example 2

The same process in Example 1 is followed except a mechanical tracking system is used instead of an optical tracking system. A mechanical tracking system with a probe is used to collect points on the exposed femur and neck to provide an initial estimate for the registration. The mechanical tracking system probe is then attached to the ultrasonic probe and ultrasonic sensor to track the position and orientation of the ultrasound probe and sensors. The surgeon uses the tracked ultrasonic probe and sensor as performed in Example 1 to collect the points, bone volume, or bone outline to accurately complete registration.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An ultrasonic system for determination of bone orientation comprising:
a base system;
a tracking system;
a plurality of ultrasonic sensors tracked by said tracking system; and
software operating on a computer to create a registration matrix model for a bone in a three dimensional workspace relative to said base system using an absence of an ultrasonic reflection from said plurality of ultrasonic sensors to build the registration model of the bone.

2. The ultrasonic system of claim 1 wherein said absence of the ultrasonic reflection is used to identify a location and an orientation of a set of conic volumes in order to build an estimate of the location and the orientation of the bone relative to the base system.

3. The ultrasonic system of claim 1 wherein said base system is registered to said bone to perform functions comprising at least one of a robotic cutting system and a navigation system.

4. The ultrasonic system of claim 1 wherein said tracking system is used to determine a point or a series of points in space, which in turn can be fixed upon an object.

5. The ultrasonic system of claim 1 wherein said tracking system includes at least one of a mechanical linkage, optical cameras, electromagnetic (EM) sensing, or a tracking technology based on the interaction of markers and cameras.

6. The ultrasonic system of claim 1 wherein said plurality of ultrasonic sensors are capable of capturing a conic ultrasonic reflection from a synchronized ultrasonic source.

7. The ultrasonic system of claim 1 wherein said plurality of ultrasonic sensors are capable of detection of at least one of ultrasonic sound waves, detection of a unidirectional ultrasonic cone while pointing in a specific orientation, or detecting a reflection of ultrasonic sound waves off a target bone within a patient.

8. The ultrasonic system of claim 1 further comprising an algorithm calculated by said software to convert an ultrasonic sensor output from one or more of said plurality of ultrasonic sensors into an oriented conic volume when coupled with a positional output from said tracking system.

9. The ultrasonic system of claim 1 further comprising an attachment mechanism between said plurality of ultrasonic sensors and a plurality of tracking system markers such that said plurality of ultrasonic sensors are tracked by said tracking system.

10. The ultrasonic system of claim 9 wherein said attachment mechanism is a rigid frame capable of holding said plurality of ultrasonic sensors and said plurality of tracking system markers in a fixed position relative to each other.

11. A process of using the system of claim 1 for ultrasonic determination of bone orientation, the process comprising:
registering said base system to the bone;
tracking said plurality of ultrasonic sensors by said tracking system;
utilizing an algorithm calculated by software executed on a computer to convert a set of positions and orientations of said plurality of ultrasonic sensors to a plurality of markers fixed to said plurality ultrasonic sensors, where a set of three dimensional (3-D) locations of said plurality of markers are attached to said plurality of ultrasonic sensors that fix the relative position and orientation of said plurality of markers to the set of positions and orientations of said plurality of ultrasonic sensors; and
said algorithm using the set of 3-D locations and the relative orientation and position of said plurality of markers tracked by the tracking system to calculate a second set of positions and orientations of ultrasonic oriented rays or ultrasonic cones generated by said plurality of ultrasonic sensors, where wherein said algorithm uses the second set of positions and orientations to track a third set of positions and orientations of the bone within a patient in 3-D space.

12. The process of claim 11 wherein said algorithm utilizes the tracked orientation and position of said plurality of ultrasonic sensors to create a solid volume model of the bone by adding tracked conic regions determined with the second set of positions and orientations, where wherein once the tracked conic regions are added, said algorithm calculates a subtraction volume by subtracting oriented conic volumes with no ultrasonic reflection, and wherein the algorithm determines a solid volume and a surface model of the bone remaining once the oriented conic volumes with no ultrasonic reflection have been subtracted with from the relative position and orientation of the bone in the 3-D space.

13. The process of claim 12 wherein said algorithm converts the surface model of the bone into a registration model.

14. The process of claim 13 wherein said algorithm converts the registration model into a registration of the surface model of the bone relative to the base system.

15. The process of claim 14 wherein said algorithm converts the registration model into a registration of the surface model of the bone relative to the base system by calculating a position of the bone based movement of the bone relative to the tracking system.

16. A process of preparing for a surgical procedure on a bone within a patient by calculating a position and an orientation of the bone within a measurement volume relative to a base system comprising:
measuring a distance to the bone in a conic region emanating from an ultrasonic sensor at a distance where an orientation and a volume of the conic region is known;
moving the conic region of the ultrasonic sensor within the measurement volume;
generating a first sweep motion to measure the distance within the conic region that intersects with the solid volume of the bone within the measurement volume;
generating a second sweep motion of said ultrasonic sensor not to measure the distance of the solid volume of the bone when the solid volume is not in the conic region;
determining the position and the orientation of said ultrasonic sensor within the measurement volume during measurement;
determining the position and the orientation of the measurement volume and the position and the orientation of the bone with respect to the base system; and
communicating to a surgeon the position and the orientation of the bone preparing for the surgical procedure on the bone within the patient.

17. The process of claim 16 wherein the measurement volume is determined by:
totaling the conic region for all collected data;
subtracting from the conic region for all collected data, the conic region of measure from the measurement volume when no distance to a solid volume is measured to determine a remaining volume;
utilizing the remaining volume to locate and orient a surface model that represents the solid volume of the bone that was located within the measurement volume during measurement; and
calculating the position and the orientation of the surface model of the solid volume within the measurement volume relative to the base system.

18. The process of claim 17 wherein the collected measurement data is determined by
moving a sweeping beam of said ultrasonic sensor along a shaft of the bone from different orientations about the shaft; and
calculating a complete result by sweeping around the bone by 180+2* cone angle during the data collection process or a partial result by using only two orientations about the shaft of the bone that are ninety degrees apart.
